(19) United States
(12) Patent Application Publication
(10) Pub. No.: US 2021/0100771 A1
(43) Pub. Date: Apr. 8, 2021

(54) NEW CANNABIS TABLET FORMULATIONS AND COMPOSITIONS AND METHODS OF MAKING THE SAME

(71) Applicant: Canopy Growth Corporation, Smiths Falls (CA)

(72) Inventor: Kurt LEVY, Idaho Springs, CO (US)

(21) Appl. No.: 17/123,866

(22) Filed: Dec. 16, 2020

Related U.S. Application Data
(63) Continuation of application No. 16/320,557, filed on Jan. 25, 2019.
(60) Provisional application No. 62/366,517, filed on Jul. 25, 2016.

(51) Int. Cl. A61K 31/352; A61K 9/20; A61K 47/38; A61K 47/02; A61K 9/14; A61K 9/00; A61K 47/10; A61K 9/16; A61K 31/01

(52) U.S. Cl. CPC A61K 31/352; A61K 9/2054; A61K 47/38; A61K 47/02; A61K 31/01; A61K 9/146; A61K 9/0053; A61K 47/10; A61K 9/1652; A61K 9/2095

(57) ABSTRACT

Disclosed herein are compositions comprising an oil having at least one cannabinoid and at least one solid powder and methods of making and using the same. In one embodiment, the compositions disclosed herein are suitable for making cannabinoid containing tablets by pressing a composition of this disclosure with one or more formulating agents, such as binders, fillers, bulking agents, excipients, etc.

### CROSS REFERENCE TO RELATED APPLICATIONS

This application is a continuation of U.S. patent application Ser. No. 16/320,557, filed Jan. 25, 2019, which is a national stage application under 35 U.S.C. 371 and claims the benefit of PCT Application No. PCT/US2017/043808 having an international filing date of 25 Jul. 2017, which designated the United States, which PCT application claims priority to U.S. Provisional Application Ser. No. 62/366,517 filed on Jul. 25, 2016, the disclosures of each of which are incorporated herein by reference in their entireties .

### TECHNICAL FIELD

This disclosure relates to the cannabis industry. In particular, this disclosure relates to compositions and formulations which are suitable for making tablets along with methods for making and using the same.

### BACKGROUND

The word “cannabis” refers to a genus of flowering plants. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

According to some accounts, cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

Cannabinoids are of particular interest for research and commercialization. Most extractions of cannabis plant matter aim to extract cannabinoids, particularly tetrahydrocannabinol (THC). THC is useful for relieving pain, treating glaucoma, and relieving nausea. THC is also gaining immense popularity as a recreational drug substance. Usually, cannabinoids are extracted from the cannabis plant as part of a crude mixture, combined with other chemical compounds found in the cannabis plant.

Cannabinoid extracts are used in a number of applications and methods of administration. However, many times the extract is a liquid with a thick, viscous consistency making the extract difficult to work with. A relatively dry form of cannabis known as “shatter” often has other compounds that may effective the activity of the desired cannabinoid or cannabinoids. Shatter still lacks versatility for administration and formulation purposes as well.

Despite developments in preparing extractions of cannabis, very little progress has been made in formulating commercial tablets or preparing compositions suitable for making tablets. Making tablets with cannabis extracts is complicated because the chemical variability across different cannabis plants makes it difficult to determine how to introduce cannabis extracts into the tablets. As a result, the edible cannabis industry faces significant problems formulating tablets with the desired physical and chemical properties. Accordingly, there exists a need for new methods of making compressed (e.g., tablet) compositions.

### DETAILED DESCRIPTION

Disclosed herein are new methods of making tablets comprising at least one cannabinoid. In one embodiment, the cannabinoid is purified. In one embodiment, the tablet comprises a terpene. In one embodiment, the terpene is purified.

Disclosed herein are new tablet compositions comprising at least one cannabinoid. In one embodiment, the cannabinoid is purified. In one embodiment, the tablet compositions comprises a terpene. In one embodiment, the terpene is purified.

Disclosed herein are new compositions (e.g., sprayable formulations and powders), which are suitable for use in methods of making tablet compositions comprising at least one cannabinoid. In one embodiment, the cannabinoid is purified. In one embodiment, the composition comprises a terpene. In one embodiment, the terpene is purified.

Disclosed herein is a composition comprising:
an oil having at least one cannabinoid; and
at least one solid powder.

Disclosed herein is a composition comprising:
at least one cannabinoid; and
a solid powder; wherein the at least one cannabinoid is evenly distributed on the solid powder, such that comparison samples of the composition show less than about 10% variation in the concentration of the at least one cannabinoid.

In one embodiment, the composition comprises a comparison samples of the composition show less than about 0-5% variation in the concentration of the at least one cannabinoid.

In one embodiment, the composition comprises a comparison samples of the composition show less than about 0-2.5% variation in the concentration of the at least one cannabinoid.

In one embodiment, the composition comprises a comparison samples of the composition show less than about 0-1% variation in the concentration of the at least one cannabinoid.

In one embodiment, the composition comprises a comparison samples of the composition show less than about 0-0.5% variation in the concentration of the at least one cannabinoid.

In one embodiment, the composition comprises a comparison samples of the composition show less than about 0-0.1% variation in the concentration of the at least one cannabinoid.

As used herein, the term “cannabinoid” refers to a compound belonging to a class of secondary compounds commonly found in plants of genus cannabis. In one embodiment, the cannabinoid is found in a plant, e.g., a plant of genus cannabis, and is sometimes referred to as a phytocannabinoid. In one embodiment, the cannabinoid is found in a mammal, sometimes called a endocannabinoid. In one embodiment, the cannabinoid is made in a laboratory setting, sometimes called a synthetic cannabinoid. In one embodiment, the cannabinoid acts upon a cellular receptor, such as a G-coupled protein receptor (e.g., a serotonin receptor, a cannabinoid receptor, TRPV1, an opioid receptor, etc.) thereby causing a response on the brain or body. In one embodiment, the cannabinoid affects the activity of other compounds at one or more receptors by acting as an agonist, partial agonist, inverse agonist, antagonist, etc.

In many cases, a cannabinoid can be identified because its chemical name will include the text string “*cannabi* in the name.

Within the context of this application, where reference is made to a particular cannabinoid, each of the acid and/or decarboxylated forms are contemplated as both single molecules and mixtures.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C₄ (CBD-C₄), Cannabidivarinic Acid (CBDVA), Cannabidivarin (CBDV), Cannabidiorcol (CBD-C₁), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C₄ (THCA-C₄), Tetrahydrocannbinol C₄ (THC-C₄), Tetrahydrocannabivarinic acid (THCVA), Tetrahydrocannabivarin (THCV), Tetrahydrocannabiorcolic acid (THCA-C₁), Tetrahydrocannabiorcol (THC-C₁), Δ⁷-cis-iso-tetrahydrocannabivarin, Δ⁸-tetrahydrocannabinolic add (Δ8-THCA), Cannabivarinodiolic (CBNDVA), Cannabivarinodiol (CBNDV), Δ⁸-tetrahydrocannabinol (Δ⁸-THC), Δ⁹-tetrahydrocannabinol Δ⁹-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA),Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C₄ (CBN-C₄), Cannabivarin (CBV), Cannabino-C₂ (CBN-C₂), Cannabiorcol (CBN-C₁), Cannabinodiol (CBND), Cannabinodiolic Acid (CBNDA), Cannabinodivarin (CBDV), Cannabitriol (CBT), 10-Ethoxy-9-hydroxy-Δ^{6a}-tetrahydrocannabinol, 8,9-Dihydroxy-Δ^{6a(10a)}-tetrahydrocannabinol (8,9-Di-OH-CBT-Cs), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-Δ^{6a(10a)}-tetrahydrocannabinol (OTHC), Δ⁹-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide.

In one embodiment, the purified cannabinoid is chosen from THC, D9-THC, D8-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, or CBLVA.

As used herein, the term “THC” refers to tetrahydrocannabinol and has the following structural formula:

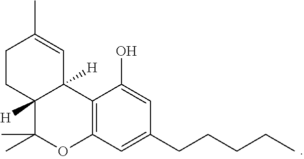 [SMILES: CCCCCc1cc(O)c2c(c1)OC(C)(C)[C@@H]1CCC(C)=C[C@@H]21]

Within the context of this disclosure, compositions comprising THC are formulated with other compounds, thereby providing previously unavailable potency, control, consistency, purity, etc.

As used herein, the term “THCA” refers to tetrahydrocannabinolic acid and has the following structural formula:

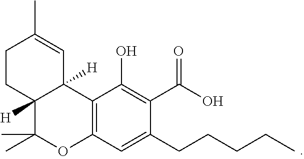 [SMILES: CCCCCc1cc2c(c(O)c1C(=O)O)[C@@H]1C=C(C)CC[C@H]1C(C)(C)O2]

Decarboxylating THCA with heat, light, etc., forms THC, D8-THC, D9-THC, and other potential cannabinoids. Within the context of this disclosure, compositions comprising THCA are formulated with other compounds, thereby providing previously unavailable potency, control, consistency, purity, etc.

As used herein, the term “THCV” refers to tetrahydrocannabivarin and has the following structural formula:

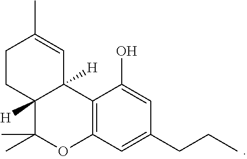 [SMILES: CCCc1cc(O)c2c(c1)OC(C)(C)[C@@H]1CCC(C)=C[C@@H]21]

Within the context of this disclosure, compositions comprising THCV are formulated with other compounds, thereby providing previously unavailable potency, control, consistency, purity, etc.

As used herein, the term “THCVA” refers to tetrahydrocannabivarinic acid and has the following structural formula:

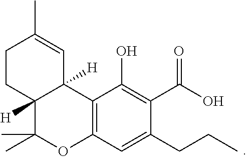 [SMILES: CCCc1cc2c(c(O)c1C(=O)O)[C@@H]1C=C(C)CC[C@H]1C(C)(C)O2]

Decarboxylating THCVA with heat, light, etc., forms THCV, D8-THCV, D9-THCV, and other possible cannabinoid derivatives. Within the context of this disclosure, compositions comprising THCVA are formulated with other compounds, thereby providing previously unavailable potency, control, consistency, purity, etc.

As used herein, the term “D8-THC” refers to delta-8-tetrahydrocannabinol and has the following structural formula:

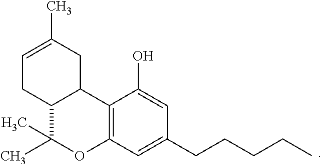

Within the context of this disclosure, compositions comprising D8-THC are formulated with other compounds, thereby providing previously unavailable potency, control, consistency, purity, etc.

As used herein, the term “D8-THCV” refers to delta-8-tetrahydrocannabivarin and has the following structural formula:

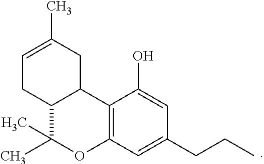

Within the context of this disclosure, compositions comprising D8-THCV are formulated with other compounds, thereby providing previously unavailable potency, control, consistency, purity, etc.

As used herein, the term “D9-THC” refers to delta-9-tetrahydrocannabinol and has the following structural formula:

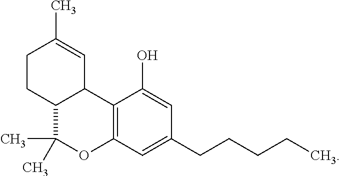

Within the context of this disclosure, compositions comprising D9-THC are formulated with other compounds, thereby providing previously unavailable potency, control, consistency, purity, etc.

As used herein, the term “D9-THCV” refers to delta-9-tetrahydrocannabivarin and has the following structural formula:

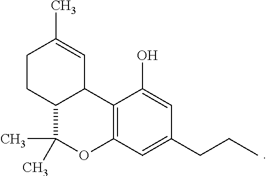

Within the context of this disclosure, compositions comprising D9-THCV are formulated with other compounds, thereby providing previously unavailable potency, control, consistency, purity, etc.

As used herein, the term “CBD” refers to cannabidiol and has the following structural formula:

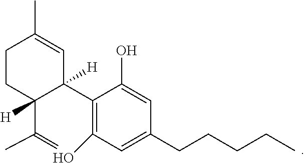 [SMILES: C=C(C)[C@@H]1CCC(C)=C[C@H]1c1c(O)cc(CCCCC)cc1O]

Within the context of this disclosure, compositions comprising CBD are formulated with other compounds, thereby providing previously unavailable potency, control, consistency, purity, etc.

As used herein, the term “CBDA” refers to cannabidiolic acid and has the following structural formula:

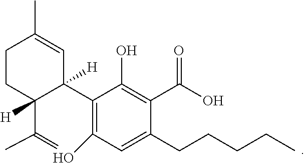 [SMILES: C=C(C)[C@@H]1CCC(C)=C[C@H]1c1c(O)cc(CCCCC)c(C(=O)O)c1O]

Decarboxylating CBDA with heat, light, etc., forms CBD and other possible cannabinoid derivatives. Within the context of this disclosure, compositions comprising CBDA are formulated with other compounds, thereby providing previously unavailable potency, control, consistency, purity, etc.

As used herein, the term “CBDV” refers to cannabidivarin and has the following structural formula:

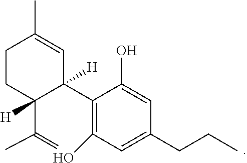 [SMILES: C=C(C)[C@@H]1CCC(C)=C[C@H]1c1c(O)cc(CCC)cc1O]

Within the context of this disclosure, compositions comprising CBDV are formulated with other compounds, thereby providing previously unavailable potency, control, consistency, purity, etc.

As used herein, the term “CBDVA” refers to cannabidivarinic acid and has the following structural formula:

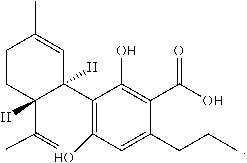 [SMILES: C=C(C)[C@@H]1CCC(C)=C[C@H]1c1c(O)cc(CCC)c(C(=O)O)c1O]

Decarboxylating CBDVA with heat, light, etc., forms CBDV and other possible cannabinoid derivatives. Within the context of this disclosure, compositions comprising CBDVA are formulated with other compounds, thereby providing previously unavailable potency, control, consistency, purity, etc.

As used herein, the term “CBC” refers to cannabichromene and has the following structural formula:

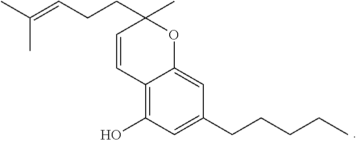 [SMILES: CCCCCc1cc(O)c2c(c1)OC(C)(CCC=C(C)C)C=C2]

Within the context of this disclosure, compositions comprising CBC are formulated with other compounds, thereby providing previously unavailable potency, control, consistency, purity, etc.

As used herein, the term “CBCA” refers to cannabichromenic acid and has the following structural formula:

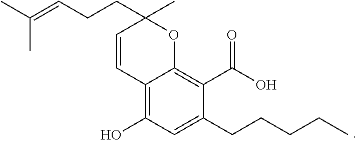 [SMILES: CCCCCc1cc(O)c2c(c1C(=O)O)OC(C)(CCC=C(C)C)C=C2]

Decarboxylating CBCA with heat, light, etc., forms CBC and other possible cannabinoid derivatives. Within the context of this disclosure, compositions comprising CBCA are formulated with other compounds, thereby providing previously unavailable potency, control, consistency, purity, etc.

As used herein, the term “CBCV” refers to cannabichromevarin and has the following structural formula:

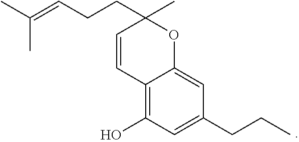 [SMILES: CCCc1cc(O)c2c(c1)OC(C)(CCC=C(C)C)C=C2]

Within the context of this disclosure, compositions comprising CBCV are formulated with other compounds, thereby providing previously unavailable potency, control, consistency, purity, etc.

As used herein, the term “CBCVA” refers to cannabichromevarinic acid and has the following structural formula:

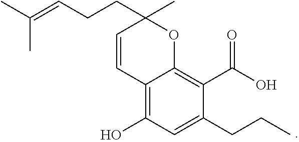 [SMILES: CCCc1cc(O)c2c(c1C(=O)O)OC(C)(CCC=C(C)C)C=C2]

Decarboxylating CBCVA with heat, light, etc., forms CBCV and other possible cannabinoid derivatives. Within the context of this disclosure, compositions comprising CBCVA are formulated with other compounds, thereby providing previously unavailable potency, control, consistency, purity, etc.

As used herein, the term “CBG” refers to cannabigerol and has the following structural formula:

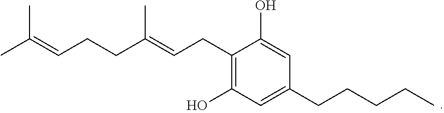 [SMILES: CCCCCc1cc(O)c(C/C=C(\C)CCC=C(C)C)c(O)c1]

Within the context of this disclosure, compositions comprising CBG are formulated with other compounds, thereby providing previously unavailable potency, control, consistency, purity, etc.

As used herein, the term “CBGA” refers to cannabigerolic acid and has the following structural formula:

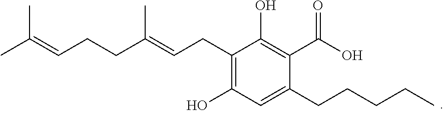 [SMILES: CCCCCc1cc(O)c(C/C=C(\C)CCC=C(C)C)c(O)c1C(=O)O]

Decarboxylating CBGA with heat, light, etc., forms CBG and other possible cannabinoid derivatives. Within the context of this disclosure, compositions comprising CBGA are formulated with other compounds, thereby providing previously unavailable potency, control, consistency, purity, etc.

As used herein, the term “CBGV” refers to cannabigerovarin and has the following structural formula:

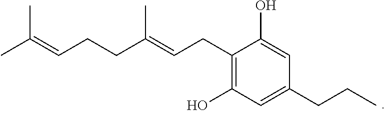 [SMILES: CCCc1cc(O)c(C/C=C(\C)CCC=C(C)C)c(O)c1]

Within the context of this disclosure, compositions comprising CBGV are formulated with other compounds, thereby providing previously unavailable potency, control, consistency, purity, etc.

As used herein, the term “CBGVA” refers to cannabigerovarinic acid and has the following structural formula:

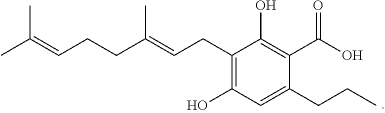 [SMILES: CCCc1cc(O)c(C/C=C(\C)CCC=C(C)C)c(O)c1C(=O)O]

Decarboxylating CBGVA with heat, light, etc., forms CBGV and other possible cannabinoid derivatives. Within the context of this disclosure, compositions comprising CBGVA are formulated with other compounds, thereby providing previously unavailable potency, control, consistency, purity, etc.

As used herein, the term “CBN” refers to cannabinol and has the following structural formula:

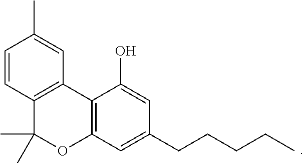 [SMILES: CCCCCc1cc(O)c2c(c1)OC(C)(C)c1ccc(C)cc1-2]

Within the context of this disclosure, compositions comprising CBN are formulated with other compounds, thereby providing previously unavailable potency, control, consistency, purity, etc.

As used herein, the term “CBNA” refers to cannabinolic acid and has the following structural formula:

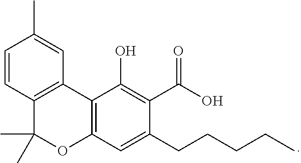 [SMILES: CCCCCc1cc2c(c(O)c1C(=O)O)-c1cc(C)ccc1C(C)(C)O2]

Decarboxylating CBNA with heat, light, etc., forms CBN and other possible cannabinoid derivatives. Within the context of this disclosure, compositions comprising CBNA are formulated with other compounds, thereby providing previously unavailable potency, control, consistency, purity, etc.

As used herein, the term “CBNV” or “CBV” refers to cannabivarin and has the following structural formula:

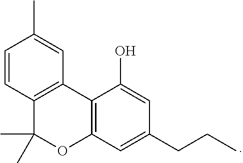 [SMILES: CCCc1cc(O)c2c(c1)OC(C)(C)c1ccc(C)cc1-2]

Within the context of this disclosure, compositions comprising CBNV are formulated with other compounds, thereby providing previously unavailable potency, control, consistency, purity, etc.

As used herein, the term “CBNVA” refers to cannabivarinic acid and has the following structural formula:

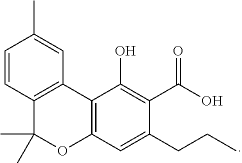 [SMILES: CCCc1cc2c(c(O)c1C(=O)O)-c1cc(C)ccc1C(C)(C)O2]

Decarboxylating CBNVA with heat, light, etc., forms CBNV and other possible cannabinoid derivatives. Within the context of this disclosure, compositions comprising CBNVA are formulated with other compounds, thereby providing previously unavailable potency, control, consistency, purity, etc.

As used herein, the term “CBND” refers to cannabinodiol and has the following structural formula:

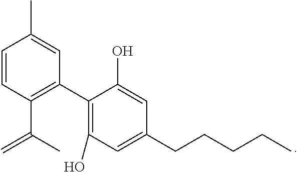 [SMILES: C=C(C)c1ccc(C)cc1-c1c(O)cc(CCCCC)cc1O]

Within the context of this disclosure, compositions comprising CBND are formulated with other compounds, thereby providing previously unavailable potency, control, consistency, purity, etc.

As used herein, the term “CBNDA” refers to cannabinodiolic acid and has the following structural formula:

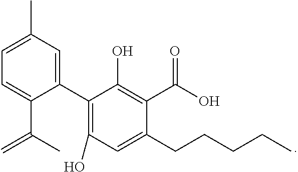 [SMILES: C=C(C)c1ccc(C)cc1-c1c(O)cc(CCCCC)c(C(=O)O)c1O]

Decarboxylating CBNDA with heat, light, etc., forms CBND and other possible cannabinoid derivatives. Within the context of this disclosure, compositions comprising CBNDA are formulated with other compounds, thereby providing previously unavailable potency, control, consistency, purity, etc.

As used herein, the term “CBNDV” refers to cannabivarinodiol and has the following structural formula:

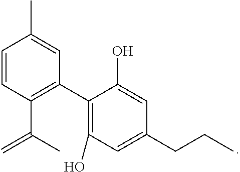 [SMILES: C=C(C)c1ccc(C)cc1-c1c(O)cc(CCC)cc1O]

Within the context of this disclosure, compositions comprising CBNDV are formulated with other compounds, thereby providing previously unavailable potency, control, consistency, purity, etc.

As used herein, the term “CBNDVA” refers to cannabivarinodiolic acid and has the following structural formula:

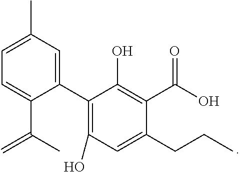 [SMILES: C=C(C)c1ccc(C)cc1-c1c(O)cc(CCC)c(C(=O)O)c1O]

Decarboxylating CBNDVA with heat, light, etc., forms CBNDV and other possible cannabinoid derivatives. Within the context of this disclosure, compositions comprising CBNDVA are formulated with other compounds, thereby providing previously unavailable potency, control, consistency, purity, etc.

As used herein, the term “CBL” refers to cannabicyclol and has the following structural formula:

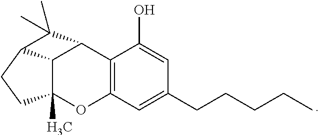

Within the context of this disclosure, compositions comprising CBL are formulated with other compounds, thereby providing previously unavailable potency, control, consistency, purity, etc.

As used herein, the term “CBLA” refers to cannabicyclolic acid and has the following structural formula:

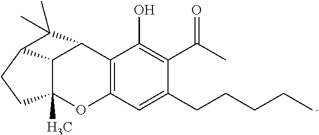

Decarboxylating CBLA with heat, light, etc., forms CBL and other possible cannabinoid derivatives. Within the context of this disclosure, compositions comprising CBLA are formulated with other compounds, thereby providing previously unavailable potency, control, consistency, purity, etc.

As used herein, the term “CBLV” refers to cannabicyclovarin and has the following structural formula:

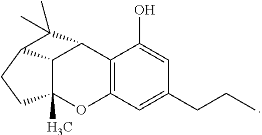

Within the context of this disclosure, compositions comprising CBLV are formulated with other compounds, thereby providing previously unavailable potency, control, consistency, purity, etc.

As used herein, the term “CBLVA” refers to cannabielvarinsoinic acid and has the following structural formula:

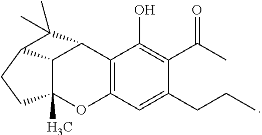

Decarboxylating CBLVA with heat, light, etc., forms CBLV and other possible cannabinoid derivatives. Within the context of this disclosure, compositions comprising CBLVA are formulated with other compounds, thereby providing previously unavailable potency, control, consistency, purity, etc.

As used herein, the term “CBE” refers to cannabielsoin and has the following structural formula:

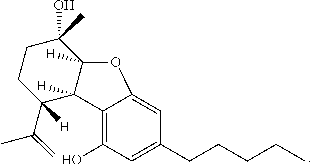

Within the context of this disclosure, compositions comprising CBE are formulated with other compounds, thereby providing previously unavailable potency, control, consistency, purity, etc.

As used herein, the term “CBEA” refers to cannabielsoic acid and has the following structural formula:

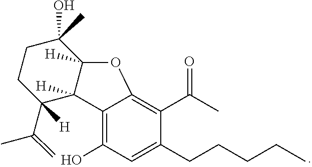

Decarboxylating CBEA with heat, light, etc., forms CBE and other possible cannabinoid derivatives. Within the context of this disclosure, compositions comprising CBEA are formulated with other compounds, thereby providing previously unavailable potency, control, consistency, purity, etc.

As used herein, the term “CBEV” refers to cannabivarinselsoin and has the following structural formula:

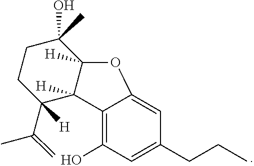

Within the context of this disclosure, compositions comprising CBEV are formulated with other compounds, thereby providing previously unavailable potency, control, consistency, purity, etc.

As used herein, the term “CBEVA” refers to cannabivarinselsoinic acid and has the following structural formula:

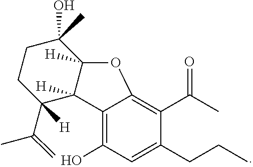

Decarboxylating CBEVA with heat, light, etc., forms CBEV and other possible cannabinoid derivatives. Within the context of this disclosure, compositions comprising CBEVA are formulated with other compounds, thereby providing previously unavailable potency, control, consistency, purity, etc.

As used herein, the term “solid” refers to a physical state of matter as compared to liquids, gases, gels, and plasmas. In one embodiment, the term “solid” refers to the state of matter at ambient temperatures. In one embodiment, the term “solid” refers to the state of matter at higher temperatures compared to the ambient temperature. In one embodiment, the term “solid” refers to the state of matter at lower temperatures compared to the ambient temperature.

As used herein, the term “powder” means a material composed of particles that are not cemented together. In one embodiment, the term powder refers to a dry, bulk solid composed of a multitude of fine particles. In one embodiment, the said particles flow freely when shaken or tilted, i.e., a “free flowing powder.” Powders are often considered a special sub-class of granular materials, although the terms powder and granular are sometimes used to distinguish separate classes of material. Within the context of this disclosure, the term powder may refer to either of both of a granular material and/or a fine, free-flowing powder. In one embodiment, the term powder refers to a granular material that has fine grain sizes.

As used herein, the term “oil having at least one cannabinoid” means a non-crystalline material that includes at least one cannabinoid, wherein that non-crystalline material exists at a variety of viscosities along the spectrum in-between a rigid crystalline solid and a liquid. For example, a gooey, syrup-like consistency, a sap-like consistency, or a waxy consistency. In one embodiment, the “oil having at least one cannabinoid” is a raw extract of at least one part of the cannabis plant.

In one embodiment, the “oil having at least one cannabinoid” comprises less than about 50% (e.g., between 0.001-49%) non-cannabinoid compounds.

In one embodiment, the “oil having at least one cannabinoid” comprises less than about 40% (e.g., between 0.001-39%) non-cannabinoid compounds.

In one embodiment, the “oil having at least one cannabinoid” comprises less than about 30% (e.g., between 0.001-29%) non-cannabinoid compounds.

In one embodiment, the “oil having at least one cannabinoid” comprises less than about 20% (e.g., between 0.001-19%) non-cannabinoid compounds.

In one embodiment, the “oil having at least one cannabinoid” comprises less than about 10% (e.g., between 0.001-9%) non-cannabinoid compounds.

In one embodiment, the “oil having at least one cannabinoid” comprises less than about 5% (e.g., between 0.001-4.9%) non-cannabinoid compounds.

In one embodiment, the “oil having at least one cannabinoid” comprises less than about 2% (e.g., between 0.001-1.9%) non-cannabinoid compounds.

In one embodiment, the “oil having at least one cannabinoid” is a raw extract of at least one part of the cannabis plant.

In one embodiment, the “oil having at least one cannabinoid” is purified and is 50-99% pure.

In one embodiment, the “oil having at least one cannabinoid” is purified and is 60-90% pure.

In one embodiment, the “oil having at least one cannabinoid” is purified and is 70-80% pure.

In one embodiment, the “oil having at least one cannabinoid” is purified and is greater than 90% pure.

In one embodiment, the “oil having at least one cannabinoid” is purified and is greater than 99% pure.

In one embodiment, the “oil having at least one cannabinoid” is purified and is greater than 99.999% pure.

In one embodiment, the “oil having at least one cannabinoid” is a purified formulation with one purified cannabinoid.

In one embodiment, the “oil having at least one cannabinoid” is a purified formulation with two purified cannabinoids.

In one embodiment, the “oil having at least one cannabinoid” is a purified formulation with three purified cannabinoids.

In one embodiment, the “oil having at least one cannabinoid” is a purified formulation with three or more purified cannabinoids.

In one embodiment, the “oil having at least one cannabinoid” is a purified formulation with a purified cannabinoid and a purified terpene.

In one embodiment, the “oil having at least one cannabinoid” is a purified formulation with a purified cannabinoid and two purified terpenes.

In one embodiment, the “oil having at least one cannabinoid” is a purified formulation with a purified cannabinoid and more than two purified terpenes.

As used within the context of this application, the term “purified” means extracted, isolated, and/or separated from other compounds, formulations, compositions, matter, and/or mass. In one embodiment, the term “purified” refers to a cannabinoid that is separated from the plant matter from which it was derived.

In one embodiment, the term “purified” refers to a cannabinoid (a “purified cannabinoid”) that is separated from other cannabinoids that were present in the plant matter from which it was derived. In one embodiment, the term “purified” refers to a cannabinoid (a “purified cannabinoid”) that is separated from terpenes that were present in the plant matter from which it was derived. In one embodiment, the term “purified” refers to a cannabinoid (a “purified cannabinoid”) that is separated from secondary compounds that were present in the plant matter from which it was derived. In one embodiment, the term “purified” refers to a cannabinoid (a “purified cannabinoid”) that is separated from all material that was present in the plant matter from which it was derived.

Within the context of this disclosure, purified compounds may be purposely formulated with other compounds at various levels of purity. For example, depending on the desired outcome, a particular cannabinoid or terpene may be formulated with other molecules when it is 60-65% pure, 65-70% pure, 70-75% pure, 75-80% pure, 80-85% pure, 85-90% pure, 90-95% pure, 95-99% pure, 99-99.9% pure, 99.9+%, or greater than 99% pure. Provided that the ingredients used for purposeful formulation are purified prior to the said purposeful formulation, the act of subsequently formulating them does render them not “purified” within the context of an ingredient list.

In one embodiment, purified means “substantially free” from other material, e.g., compounds, particles, vegetative material, plant derived substances, solvents, etc. In one example, the term “purified” refers to a compound purified from a crude extract, such as a biologically derived substance or BDS, thereby resulting in a significant difference between the purified compound and the extract. In one embodiment, substantially free means that the compound comprises no (or insignificant amounts) of other materials.

Within the context of this disclosure, where a compound comprises stereogenic centers, the term “purified” includes isolated stereoisomers and also mixtures of stereoisomers, provided that the compound having the stereoisomers is free from other compounds having different atomic connectivity.

In one embodiment, purification comprises using various solvents, e.g., ethanol, butane, methane, carbon dioxide, ice, water, steam. In one embodiment, purification comprises various techniques, e.g., chromatography, crystallization, filtration, centrifuge, etc. or various combinations of said techniques. In one embodiment, purification comprises extracting cannabinoids and other plant molecules from plants bred to express desired cannabinoid and/or terpene profiles for purity.

As used herein, the term “concentration” refers to a ratio of compounds in relation to another. In one embodiment, the concentration is expressed as the amount of moles of one compound in relation to total amount of moles a sample. In one embodiment, the concentration is expressed as the amount of moles of one compound in relation to total amount of moles a sample. In one embodiment, the concentration is expressed as the mass of one compound in relation to total volume of the sample. In one embodiment, the concentration is expressed as amount of moles of one compound in relation to total volume of the sample. In one embodiment, the concentration is expressed as the volume of one compound in relation to total volume of the sample.

Disclosed herein is a composition comprising an oil having at least one cannabinoid; at least one solid powder; and an alcohol. In one embodiment the alcohol is ethanol.

Disclosed herein is a sprayable composition, comprising at least one cannabinoid dissolved in an alcohol. In one embodiment, the composition is a solution of at least one cannabinoid dissolved in ethanol.

In one embodiment, the ethanol is present in about 20 to 80% of the mass of the oil having at least one cannabinoid.

In one embodiment, the ethanol is present in about 30 to 75% of the mass of the oil having at least one cannabinoid.

In one embodiment, the ethanol is present in about 40 to 60% of the mass of the oil having at least one cannabinoid.

In one embodiment, the ethanol is present in about 45 to 55% of the mass of the oil having at least one cannabinoid.

In one embodiment, the composition comprises ethanol in an amount detectable spectroscopically; wherein the ethanol is present in less than 0.1 mass % within the composition.

In one embodiment, the composition comprises ethanol in an amount detectable spectroscopically; wherein the ethanol is present between about 0.1-0.01 mass % within the composition.

In one embodiment, the composition comprises ethanol in an amount detectable spectroscopically; wherein the ethanol is present between about 0.01-0.001 mass % within the composition.

In one embodiment, the composition comprises ethanol in an amount detectable spectroscopically; wherein the ethanol is present between about 0.001-0.0001 mass % within the composition.

In one embodiment, the sprayable compositions disclosed herein are sprayed onto a tumbling powder.

In one embodiment of the disclosed composition, the at least one cannabinoid is a mixture of cannabinoids. In one embodiment, the mixture of cannabinoids consists of two cannabinoids. In one embodiment, the mixture of cannabinoids consists of three cannabinoids. In one embodiment, the mixture of cannabinoids consists of four cannabinoids. In one embodiment, the mixture of cannabinoids consists of five cannabinoids. In one embodiment, the mixture of cannabinoids consists of six cannabinoids. In one embodiment, the mixture of cannabinoids consists of seven cannabinoids. In one embodiment, the mixture of cannabinoids consists of eight cannabinoids. In one embodiment, the mixture of cannabinoids consists of nine cannabinoids. In one embodiment, the mixture of cannabinoids consists of ten cannabinoids. In one embodiment, the mixture of cannabinoids consists of more than ten cannabinoids.

Disclosed herein is a composition comprising at least one cannabinoid, which is suitable for making compressible dosage forms. In one embodiment, the composition is an infused powder. In one embodiment, the composition suitable for making compressible dosage forms comprises at least one solid powder. In one embodiment, the composition suitable for making compressible dose forms comprises a food.

In one embodiment, the compositions disclosed herein comprise between about 80% to about 90% food.

In one embodiment, the compositions disclosed herein comprise between about 70% to about 95% food.

Disclosed herein is a method of making a cannabinoid infused powder, comprising:
adding alcohol to an oil having at least one cannabinoid;
dissolving the oil having at least one cannabinoid into the alcohol, thereby creating a solution;
spraying the said solution onto a solid powder; and
evaporating the alcohol.

Disclosed herein is a method of making a cannabinoid infused powder, comprising:
adding ethanol to an oil having at least one cannabinoid;
dissolving the oil having at least one cannabinoid into the ethanol, thereby creating a solution;
spraying the said solution onto a solid powder;
evaporating the ethanol.

In one embodiment of the disclosed method, the oil having at least one cannabinoid includes between 40% to 99% cannabinoids by mass.

In one embodiment of the disclosed method, the oil having at least one cannabinoid includes between 50% to 90% cannabinoids by mass.

In one embodiment of the disclosed method, the oil having at least one cannabinoid includes between 55% to 80% cannabinoids by mass.

In one embodiment of the disclosed method, the oil having at least one cannabinoid includes between 60% to 70% cannabinoids by mass.

In one embodiment of the disclosed method, the ethanol is added to the oil in an amount of about 30% to 90% relative to the mass of the oil.

In one embodiment of the disclosed method, the ethanol is added to the oil in an amount of about 35% to 85% relative to the mass of the oil.

In one embodiment of the disclosed method, the ethanol is added to the oil in an amount of about 40% to 75% relative to the mass of the oil.

In one embodiment of the disclosed method, the ethanol is added to the oil in an amount of about 45% to 55% relative to the mass of the oil.

In one embodiment of the disclosed method, the oil having at least one cannabinoid is evenly distributed upon the at least one solid powder.

Disclosed herein is a method of making a tablet comprising:
adding alcohol to an oil having at least one cannabinoid;
dissolving the oil having at least one cannabinoid into the alcohol, thereby creating a solution;
spraying the said solution onto a solid powder;
evaporating the alcohol to create a powder impregnated with at least one cannabinoid;
combining the powder with at least one formulating agent to create a loose mixture of solids;
applying pressure to the loose mixture of solids to create a unified mass.

Disclosed herein is a method of making a tablet comprising:
adding ethanol to an oil having at least one cannabinoid;
dissolving the oil having at least one cannabinoid into the ethanol, thereby creating a solution;
spraying the said solution onto a solid powder;
evaporating the ethanol to create a powder impregnated with at least one cannabinoid;
combining the powder with at least one formulating agent to create a loose mixture of solids;
applying pressure to the loose mixture of solids to create a unified mass.

As used herein, the term “adding” refers to joining two or more things together. In one embodiment, adding comprises joining two compounds together. In one embodiment, adding comprises joining an alcohol and an oil.

As used herein, the term “dissolving” refers to converting the particle of a compound to a lower state of stability and volume. In one embodiment, dissolving comprises forming a solution by placing a solid into a liquid. In one embodiment, dissolving comprises forming a homogenous mixture of a liquid and oil. In one embodiment, dissolving comprises forming a homogenous mixture of an alcohol and a cannabinoid oil.

As used herein, the term “solution” refers to a mixture or formulation of two or more compounds. In one embodiment, the solution is a mixture of two or more liquids. In one embodiment, the solution is a cannabinoid oil dispersed in an alcohol.

As used herein, the term “spraying” refers to dispersing a compound or compounds into fine particles or droplets. In one embodiment, spraying comprises dispersing a liquid into a fine mist. In one embodiment, spraying comprises using a spray bottle. In one embodiment, spraying comprises forcing a compound through perforations.

As used herein, the term “evaporating” refers to converting a compound into the vapor phase. In one embodiment, evaporating comprises heating a compound. In one embodiment, evaporating comprises applying pressure. In one embodiment, evaporating comprises applying circulation of air at ambient temperature. In one embodiment, evaporating comprises turning a liquid into a gas. In one embodiment, evaporating comprises turning a solid into a gas, also known as sublimating.

As used herein, the term “combining” refers to merging, incorporating, fusing, blending, or mixing. In one embodiment, combining comprises mixing compounds to form a homogeneous mixture.

As used herein, the term “formulating agent” refers to a substance altering the physical and/or chemical properties of a compound or sample. In one embodiment, the formulating agent prevents clumping. In one embodiment, the formulating agent is an emulsifier. In one embodiment, the formulating agent adds mass to a sample.

As used herein, the term “loose mixture of solids” refers to a blend, combination, or collection of compounds in an unbound structure. In one embodiment, the loose mixture of solids is a flowable powder. In one embodiment, the loose mixture of solids refers to a collection of compounds with weak chemical compounds, e.g., van der Waals interactions.

As used herein, the term “applying pressure” refers to administering a force or forces. In one embodiment, applying pressure comprises utilizing a vacuum chamber. In one embodiment, applying pressure comprises applying a piston.

In one embodiment of the methods disclosed herein, the “formulating agent” with which the impregnated powder is combined is replaced with a food.

As used herein the term “food” means the Federal Drug Administration's definition of food.

In one embodiment, the powder impregnated with at least one cannabinoid is a flowable powder.

In one embodiment, the tablet disclosed herein comprises between about 0.1% to about 2.5% by mass THC.

In one embodiment, the tablet disclosed herein comprises between about 0.5% to about 1.5% by mass THC.

In one embodiment, the compositions disclosed herein comprise about 0.5% to 1.5% THC distributed within a solid mass comprising Microcrystalline Cellulose, Colloidal Silicon Dioxide, Sodium Starch Glycolate, and Sodium Stearyl Fumarate.

In one embodiment, the compositions disclosed herein comprise about 0.5% to 1.5% THC distributed within a solid mass comprising Microcrystalline Cellulose and Colloidal Silicon Dioxide.

In one embodiment, the compositions disclosed herein comprise about 0.1% to 2.5% THC distributed within a solid mass comprising Microcrystalline Cellulose, Colloidal Silicon Dioxide, Sodium Starch Glycolate, and Sodium Stearyl Fumarate.

In one embodiment, the compositions disclosed herein comprise about 0.1% to 2.5% THC distributed within a solid mass comprising Microcrystalline Cellulose and Colloidal Silicon Dioxide.

In one embodiment of the tablet disclosed herein, the tablet comprises at least one flavoring compound.

In one embodiment of the disclosed method of making a tablet, the method comprises applying pressure with a tablet press.

In one embodiment of the tablet disclosed herein, the tablet comprises at least one formulating agent. In one embodiment, the formulating agent is chosen from a binder, a filler, or a bulking agent.

Disclosed herein is a method of making a tablet, comprising dissolving a cannabis extract in ethanol to make a solution, then spraying the solution onto a mixture of microcrystalline cellulose and silicon dioxide, then evaporating the ethanol to create a cannabis infused powder, which is combined with a bulking agent, then pressed to create a tablet. In one embodiment, the bulking agent is a food, such as a sugar or flavoring agent.

As used herein, the term “sugar” refers to a compound used by organisms to store energy. Sugar is often used in food products as a sweetener and may provide other benefits, e.g., preservative, texture modifier, flavoring agent, bulking agent, etc. In one embodiment, the sugar is a carbohydrate. In one embodiment, the sugar is a monosaccharide. In one embodiment, the sugar is a disaccharide. In one embodiment, the sugar is a oligosaccharide. In one embodiment, the sugar is a short composed of carbon, hydrogen, and oxygen. In one embodiment, the sugar has the formula CₙH₂ₙOₙ, wherein n is an integer. In one embodiment, n is 3. In one embodiment, n is 4. In one embodiment, n is 5. In one embodiment, n is 6. In one embodiment, n is 7.

Within the context of this disclosure, the term sugar may also refer to a number of naturally occurring or synthetic compounds imparting sweetness. For example, maltodextrin, sorbitol, stevia, mannitol, aspartame, sucralose, isomalt, xylitol, etc.

As used herein, the term “flavoring agent” refers to a compound or mixture of compounds imparting or modifying a taste. In one embodiment, the flavoring agent is sugar. In one embodiment, the flavoring agent is salt. In one embodiment, the flavoring agent is a bitter blocker. In one embodiment, the flavoring agent is vanilla. In one embodiment, the flavoring agent is citrus. In one embodiment, the flavoring agent is lemon. In one embodiment, the flavoring agent is orange. In one embodiment, the flavoring agent is chocolate. In one embodiment, the flavoring agent is fruit. In one embodiment, the flavoring agent is strawberry. In one embodiment, the flavoring agent is banana. In one embodiment, the flavoring agent is cherry. In one embodiment, the flavoring agent is blueberry. In one embodiment, the flavoring agent is a terpene. In one embodiment, the flavoring agent is limonene. In one embodiment, the flavoring agent is linalool. In one embodiment, the flavoring agent is Beta-Caryophyllene.

As used herein, the term “terpene” refers to a compound built on an isoprenoid structure or produced by combining isoprene units, 5 carbon structures. Terpenes are also associated with producing smell in plants where terpenes are part of a class of secondary compounds. In one embodiment, the terpene is a hydrocarbon.

Within the context of this disclosure, the term “terpene” does not necessarily require 5 carbons or multiples of 5 carbons. It is understood that a reaction with isoprene units does not always result in a terpene comprising all the carbon atoms.

Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Within the context of this disclosure, the term terpene includes the α- (alpha), β- (beta), γ- (gamma), oxo-, isomers, or any combinations thereof.

Examples of terpenes within the context of this disclosure include: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha, Bisabolol, Borneol, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Caryophyllene (Beta-Caryophyllene), Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), lonol (3-oxo-alpha-ionol) (Beta-Ionol), Ionone (Alpha-Ionone) (Beta-Ionone), 1psdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In one embodiment, the terpene is chosen from Limonene, Nerolidol, Beta-Myrcene, Linalool, Alpha-Caryophyllene, Beta-Caryophyllene, Alpha-Pinene, Beta-Pinene, Alpha-Bisabolol, Delta-3-Carene, Borneol, p-Cymene, Eucalyptol, Alpha-Humulene, Alpha-Terpineol, Terpinolene, Pulegone, Camphene, or Geraniol.

In one embodiment, the mixture of microcrystalline cellulose and silicon dioxide additionally comprises Sodium Starch Glycolate and Sodium Stearyl Fumarate.

As used herein, the term “microcrystalline cellulose” refers to an inert polymer. In one embodiment, microcrystalline cellulose is derived from plants. In one embodiment, microcrystalline cellulose is derived from wood pulp. In one embodiment, microcrystalline cellulose is an excipient.

As used herein, the term “silicon dioxide” refers to an oxide of silicon with the chemical formula SiO₂. In one embodiment, silicon dioxide is found in quartz. In one embodiment, silicon dioxide is found in sand.

As used herein, the term “sodium starch glycolate” refers to a sodium salt of carboxymethyl ester. In one embodiment, sodium starch glycolate is an excipient. In one embodiment, sodium starch glycolate is a disintegrant. In one embodiment, sodium starch glycolate is a suspending agent. In one embodiment, sodium starch glycolate is a gelling agent. In one embodiment, sodium starch glycolate absorbs water.

As used herein, the term “sodium stearyl fumarate” refers to a water soluble lubricant. In one embodiment, sodium stearyl fumarate is inert. In one embodiment, sodium stearyl fumarate is hydrophilic. In one embodiment, sodium stearyl fumarate is a tablet lubricant.

In one embodiment, the cannabis infused powder is mixed with a solid food powder (such as sugar or a flavoring agent). In one embodiment, the cannabis infused powder is mixed with a solid food powder (such as sugar or a flavoring agent) to create a loose powder suitable for making tablets and having a THC concentration of about 1 to 1.5%. In one embodiment, the loose powder is pressed into tablets.

### EXAMPLES

The following examples are for exemplary purposes and are not meant to limit any potential embodiments within the context of this disclosure.

### Example 1

10 g of a cannabinoid oil and 15 mL of an alcohol were combined to make a homogenous alcoholic solution. 90 g of a powder was measured out and spread over a flat surface. The alcoholic solution was then loaded into a sprayer and sprayed over the powder. The alcohol was then evaporated from the powder leaving only the powder and the cannabinoid oil. The powder was then dried and mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 2

10 g of a cannabinoid oil comprising THC was combined with 15 mL of ethanol to make a homogenous ethanolic solution. 90 g of a powder was measured out and spread over a flat surface. The ethanolic solution was loaded into a sprayer and sprayed over the powder. The powder was then placed in an oven to evaporate the ethanol. The powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 3

10 g of a cannabinoid oil comprising CBD was combined with 15 mL of ethanol to make a homogenous ethanolic solution. 90 g of a powder was measured out and spread over a flat surface. The ethanolic solution was loaded into a sprayer and sprayed over the powder. The powder was then placed in an oven to evaporate the ethanol. The powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 4

10 g of a cannabinoid oil comprising THC and CBD was combined with 15 mL of ethanol to make a homogenous ethanolic solution. 90 g of a powder was measured out and spread over a flat surface. The ethanolic solution was loaded into a sprayer and sprayed over the powder. The powder was then placed in an oven to evaporate the ethanol. The powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 5

15 g of a cannabinoid oil comprising THC and Linalool was combined with 20 mL of ethanol to make a homogenous ethanolic solution. 90 g of a powder was measured out and spread over a flat surface. The ethanolic solution was loaded into a sprayer and sprayed over the powder. The powder was then placed in an oven to evaporate the ethanol. The powder was then mixed resulting in a homogenous, flowable powder. The mass percent was calculated by dividing the mass of the cannabinoid oil by the total mass of the dried powder.

(15 g cannabinoid oil)/(15 g cannabinoid oil+90 g powder)×100%=14%

### Example 6

15 g of a cannabinoid oil comprising THC, THCA, CBDA, CBD, and CBN was combined with 25 mL of ethanol to make a homogenous ethanolic solution. 100 g of a powder was measured out and spread out on a flat surface. The ethanolic solution was loaded into a sprayer and sprayed over a powder. The powder was then placed in an oven to evaporate the ethanol. The powder was then mixed resulting in a homogenous, flowable powder. The mass percent was calculated by dividing the mass of the cannabinoid oil by the total mass of the dried powder.

(15 g cannabinoid oil)/(15 g cannabinoid oil+100 g powder)×100%=13%

### Example 7

5 g of a cannabinoid oil comprising THC was combined with 10 mL of ethanol to make a homogenous ethanolic solution. 80 g of a powder comprising microcrystalline cellulose and SiO₂ was measured and spread out over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+80 g powder)×100%=5.9%

### Example 8

10 g of a cannabinoid oil comprising CBD was combined with 15 mL of ethanol to make a homogenous ethanolic solution. 90 g of a powder comprising microcrystalline cellulose and SiO₂ was measured and spread out over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 9

15 g of a cannabinoid oil comprising THC and CBD was combined with 25 mL of ethanol to make a homogenous ethanolic solution. 85 g of a powder comprising microcrystalline cellulose and SiO₂ was measured and spread out over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 10

20 g of a cannabinoid oil comprising THC and Linalool was combined with 35 mL of ethanol to make a homogenous ethanolic solution. 80 g of a powder comprising microcrystalline cellulose and SiO₂ was measured and spread out over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 11

15 g of a cannabinoid oil comprising THC, THCA, CBDA, CBD, and CBN was combined with 20 mL of ethanol to make a homogenous ethanolic solution. 85 g of a powder comprising microcrystalline cellulose and SiO₂ was measured and spread out over a flat surface. The ethanolic solution was loaded into a sprayer and sprayed over the powder. The sprayed powder was then placed in an oven to evaporate the ethanol The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 12

20 g of a cannabinoid oil comprising THC was combined with 30 mL of ethanol to make a homogenous ethanolic solution. 90 g of a powder comprising sorbitol and a microcrystalline cellulose, e.g., the microcrystalline cellulose under the Diapac brand was measured out and spread out over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+90 g powder)×100%=18%

### Example 13

5 g of a cannabinoid oil comprising CBD was combined with 15 mL of ethanol to make a homogenous ethanolic solution. 90 g of a powder comprising sorbitol and a microcrystalline cellulose, e.g., the microcrystalline cellulose under the Diapac brand was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+90 g powder)×100%=5.3%

### Example 14

5 g of a cannabinoid oil comprising THC and CBD was combined with 15 mL of ethanol to make a homogenous ethanolic solution. 100 g of a powder comprising sorbitol and a microcrystalline cellulose, e.g., the microcrystalline cellulose under the Diapac brand was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+100 g powder)×100%=4.8%

### Example 15

10 g of a cannabinoid oil comprising THC and Linalool was combined with 20 mL of ethanol to make a homogenous ethanolic solution. 80 g of a powder comprising sorbitol and a microcrystalline cellulose, e.g., the microcrystalline cellulose under the Diapac brand was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+80 g powder)×100%=11%

### Example 16

10 g of a cannabinoid oil comprising THC, THCA, CBDA, CBD, and CBN was combined with 20 mL of ethanol to make a homogenous ethanolic solution. 90 g of a powder comprising sorbitol and a microcrystalline cellulose, e.g., the microcrystalline cellulose under the Diapac brand was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 17

15 g of a cannabinoid oil comprising THC was combined with 15 g of ethanol to make a homogenous ethanolic solution of a 1:1 ratio of the cannabinoid oil to the ethanol by mass. 85 g of a powder comprising microcrystalline cellulose and SiO₂ was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 18

15 g of a cannabinoid oil comprising CBD was combined with 15 g of ethanol to make a homogenous ethanolic solution of a 1:1 ratio of the cannabinoid oil to the ethanol by mass. 85 g of a powder comprising microcrystalline cellulose and SiO₂ was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 19

15 g of a cannabinoid oil comprising THC and CBD was combined with 15 g of ethanol to make a homogenous ethanolic solution of a 1:1 ratio of the cannabinoid oil to the ethanol by mass. 85 g of a powder comprising microcrystalline cellulose and SiO₂ was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 20

15 g of a cannabinoid oil comprising THC and Linalool was combined with 15 g of ethanol to make a homogenous ethanolic solution of a 1:1 ratio of the cannabinoid oil to the ethanol by mass. 85 g of a powder comprising microcrystalline cellulose and SiO₂ was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 21

15 g of a cannabinoid oil comprising THC, THCA, CBDA, CBD, and CBN was combined with 15 g of ethanol to make a homogenous ethanolic solution of a 1:1 ratio of the cannabinoid oil to the ethanol by mass. 85 g of a powder comprising microcrystalline cellulose and SiO₂ was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 22

10 g of a cannabinoid oil comprising THC was combined with 10 g of ethanol to make a homogenous ethanolic solution of a 1:1 ratio of the cannabinoid oil to the ethanol by mass. 90 g of a powder comprising sorbitol and microcrystalline cellulose, e.g., microcrystalline cellulose known as Diapac, was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 23

10 g of a cannabinoid oil comprising CBD was combined with 10 g of ethanol to make a homogenous ethanolic solution of a 1:1 ratio of the cannabinoid oil to the ethanol by mass. 90 g of a powder comprising sorbitol and microcrystalline cellulose, e.g., microcrystalline cellulose known as Diapac, was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 24

10 g of a cannabinoid oil comprising THC and CBD was combined with 10 g of ethanol to make a homogenous ethanolic solution of a 1:1 ratio of the cannabinoid oil to the ethanol by mass. 90 g of a powder comprising sorbitol and microcrystalline cellulose, e.g., microcrystalline cellulose known as Diapac, was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 25

10 g of a cannabinoid oil comprising THC and Linalool was combined with 10 g of ethanol to make a homogenous ethanolic solution of a 1:1 ratio of the cannabinoid oil to the ethanol by mass. 90 g of a powder comprising sorbitol and microcrystalline cellulose, e.g., microcrystalline cellulose known as Diapac, was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 26

10 g of a cannabinoid oil comprising THC, THCA, CBDA, CBD, and CBN was combined with 10 g of ethanol to make a homogenous ethanolic solution of a 1:1 ratio of the cannabinoid oil to the ethanol by mass. 90 g of a powder comprising sorbitol and microcrystalline cellulose, e.g., microcrystalline cellulose known as Diapac, was measured out and spread over a flat surface. The ethanolic solution was loaded into a sprayer and sprayed over the powder. The sprayed powder was then placed in an oven to evaporate the ethanol. The dried powder was then mixed resulting in a homogenous, flowable powder. The mass percent was calculated by dividing the mass of the cannabinoid oil by the total mass of the dried powder.

(10 g cannabinoid oil)/(10 g cannabinoid oil+90 g powder)×100%=10%

### Example 27

20 g of a cannabinoid oil comprising THC was combined with 20 g of ethanol to make a homogenous ethanolic solution of a 1:1 ratio of the cannabinoid oil to the ethanol by mass. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 28

20 g of a cannabinoid oil comprising THC and CBD was combined with 20 g of ethanol to make a homogenous ethanolic solution of a 1:1 ratio of the cannabinoid oil to the ethanol by mass. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 29

20 g of a cannabinoid oil comprising THC and CBD was combined with 20 g of ethanol to make a homogenous ethanolic solution of a 1:1 ratio of the cannabinoid oil to the ethanol by mass. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 30

20 g of a cannabinoid oil comprising THC and Linalool was combined with 20 g of ethanol to make a homogenous ethanolic solution of a 1:1 ratio of the cannabinoid oil to the ethanol by mass. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 31

20 g of a cannabinoid oil comprising THC, THCA, CBDA, CBD, and CBN was combined with 20 g of ethanol to make a homogenous ethanolic solution of a 1:1 ratio of the cannabinoid oil to the ethanol by mass. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 32

5 g of a cannabinoid oil comprising THC was combined with 5 g of ethanol to make a homogenous ethanolic solution of a 1:1 ratio of the cannabinoid oil to the ethanol by mass. 95 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+95 g powder)×100%=5%

### Example 33

5 g of a cannabinoid oil comprising CBD was combined with 5 g of ethanol to make a homogenous ethanolic solution of a 1:1 ratio of the cannabinoid oil to the ethanol by mass. 95 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+95 g powder)×100%=5%

### Example 34

5 g of a cannabinoid oil comprising THC and CBD was combined with 5 g of ethanol to make a homogenous ethanolic solution of a 1:1 ratio of the cannabinoid oil to the ethanol by mass. 95 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+95 g powder)×100%=5%

### Example 35

5 g of a cannabinoid oil comprising THC and Linalool was combined with 5 g of ethanol to make a homogenous ethanolic solution of a 1:1 ratio of the cannabinoid oil to the ethanol by mass. 95 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+95 g powder)×100%=5%

### Example 36

5 g of a cannabinoid oil comprising THC, THCA, CBD, CBDA, and CBN was combined with 5 g of ethanol to make a homogenous ethanolic solution of a 1:1 ratio of the cannabinoid oil to the ethanol by mass. 95 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(5 g cannabinoid oil)/(5 g cannabinoid oil+95 g powder)×100%=5%

### Example 37

4 g of a cannabinoid oil comprising THC was combined with 10 g of ethanol. 96 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 38

4 g of a cannabinoid oil comprising CBD was combined with 10 g of ethanol. 96 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 39

4 g of a cannabinoid oil comprising THC and CBD was combined with 10 g of ethanol. 96 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 40

4 g of a cannabinoid oil comprising THC and Linalool was combined with 10 g of ethanol. 96 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 41

4 g of a cannabinoid oil comprising THC, THCA, CBD, CBDA, and CBN was combined with 10 g of ethanol. 96 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 42

4 g of a cannabinoid oil comprising THC was combined with 10 g of ethanol. 96 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 43

4 g of a cannabinoid oil comprising CBD was combined with 10 g of ethanol. 96 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 44

4 g of a cannabinoid oil comprising THC and CBD was combined with 10 g of ethanol. 96 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 45

4 g of a cannabinoid oil comprising THC and Linalool was combined with 10 g of ethanol. 96 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 46

4 g of a cannabinoid oil comprising THC, THCA, CBD, CBDA, and CBN was combined with 10 g of ethanol. 96 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(4 g cannabinoid oil)/(4 g cannabinoid oil+96 g powder)×100%=4%

### Example 47

8 g of a cannabinoid oil comprising THC was combined with 15 g of ethanol. 92 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 48

8 g of a cannabinoid oil comprising CBD was combined with 15 g of ethanol. 92 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 49

8 g of a cannabinoid oil comprising THC and CBD was combined with 15 g of ethanol. 92 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 50

8 g of a cannabinoid oil comprising THC and Linalool was combined with 15 g of ethanol. 92 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 51

8 g of a cannabinoid oil comprising THC, THCA, CBD, CBDA, and CBN was combined with 15 g of ethanol. 92 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 52

8 g of a cannabinoid oil comprising THC was combined with 20 g of ethanol. 92 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 53

8 g of a cannabinoid oil comprising CBD was combined with 20 g of ethanol. 92 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 54

8 g of a cannabinoid oil comprising THC and CBD was combined with 20 g of ethanol. 92 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 55

8 g of a cannabinoid oil comprising THC and Linalool was combined with 20 g of ethanol. 92 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 56

8 g of a cannabinoid oil comprising THC, THCA, CBD, CBDA, and CBN was combined with 20 g of ethanol. 92 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(8 g cannabinoid oil)/(8 g cannabinoid oil+92 g powder)×100%=8%

### Example 57

15 g of a cannabinoid oil comprising THC was combined with 30 g of ethanol. 85 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 58

15 g of a cannabinoid oil comprising CBD was combined with 30 g of ethanol. 85 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 59

15 g of a cannabinoid oil comprising THC and CBD was combined with 30 g of ethanol. 85 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 60

15 g of a cannabinoid oil comprising THC and Linalool was combined with 30 g of ethanol. 85 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 61

15 g of a cannabinoid oil comprising THC, THCA, CBD, CBDA, and CBN was combined with 30 g of ethanol. 85 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 62

15 g of a cannabinoid oil comprising THC was combined with 30 g of ethanol. 85 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 63

15 g of a cannabinoid oil comprising CBD was combined with 30 g of ethanol. 85 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 64

15 g of a cannabinoid oil comprising THC and CBD was combined with 30 g of ethanol. 85 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 65

15 g of a cannabinoid oil comprising THC and Linalool was combined with 30 g of ethanol. 85 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 66

15 g of a cannabinoid oil comprising THC, THCA, CBD, CBDA, and CBN was combined with 30 g of ethanol. 85 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(15 g cannabinoid oil)/(15 g cannabinoid oil+85 g powder)×100%=15%

### Example 67

20 g of a cannabinoid oil comprising THC was combined with 30 g of ethanol. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 68

20 g of a cannabinoid oil comprising CBD was combined with 30 g of ethanol. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 69

20 g of a cannabinoid oil comprising THC and CBD was combined with 30 g of ethanol. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 70

20 g of a cannabinoid oil comprising THC and Linalool was combined with 30 g of ethanol. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 71

20 g of a cannabinoid oil comprising THC, THCA, CBD, CBDA, and CBN was combined with 30 g of ethanol. 80 g of a powder comprising microcrystalline cellulose and SiO₂ was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 72

20 g of a cannabinoid oil comprising THC was combined with 30 g of ethanol. 80 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 73

20 g of a cannabinoid oil comprising CBD was combined with 30 g of ethanol. 80 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 74

20 g of a cannabinoid oil comprising THC and CBD was combined with 30 g of ethanol. 80 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 75

20 g of a cannabinoid oil comprising THC and Linalool was combined with 30 g of ethanol. 80 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

### Example 76

20 g of a cannabinoid oil comprising THC, THCA, CBD, CBDA, and CBN was combined with 30 g of ethanol. 80 g of a powder comprising sorbitol and microcrystalline cellulose, e.g., microcrystalline cellulose known as Diapac, was measured out and spread over a flat surface. The ethanolic solution was loaded into a sprayer and sprayed over the powder. A fan circulating air was used to evaporate the ethanol. The dried powder was then mixed resulting in a homogenous, flowable powder. The mass percent was calculated by dividing the mass of the cannabinoid oil by the total mass of the dried powder.

(20 g cannabinoid oil)/(20 g cannabinoid oil+80 g powder)×100%=20%

Although the present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

1-23. (canceled)

24. A composition comprising:
at least one cannabinoid;
a solid powder comprising Microcrystalline Cellulose, Colloidal Silicon Dioxide, Sodium Starch Glycolate, or Sodium Stearyl Fumarate; and
a formulating agent comprising a binder, a filler, or a bulking agent.

25. The composition according to claim 24, wherein the solid powder comprises Colloidal Silicon Dioxide.

26. The composition according to claim 24, wherein the formulating agent comprises at least one sugar.

27. The composition according to claim 26, wherein the at least one sugar comprises maltodextrin, sorbitol, stevia, mannitol, aspartame, sucralose, isomalt, or xylitol.

28. The composition according to claim 26, wherein the at least one sugar is mal todextrin.

62. The composition according to claim 26, wherein the at least one sugar is stevi a.

30. The composition according to claim 26, wherein the at least one sugar is xylitol.

31. The composition according to claim 26, wherein the at least one sugar is maltodextrin, stevia and xylitol.

32. The composition according to claim 24, wherein the at least one cannabinoid comprises D9.-THC, DS-THC, THCA, THCV, D8-THCV, D9-THCV, THCVA, CBD, CBDA, CBDV, CBDVA, CBC, CBCA, CBCV, CBCVA, CBG, CBGA, CBGV, CBGVA, CBN, CRNA, CBNV, CBNVA, CBND, CBNDA, CBNDV, CBNDVA, CBE, CBEA, CBEV, CBEVA, CBL, CBLA, CBLV, or CBLVA.

33. The composition according to claim 32, wherein the at least one cannabinoid is THC,

34. The composition according to claim 32, wherein the at least one cannabinoid is CBD.

35. The composition according to claim 32, wherein the at least one cannabinoid is THC and CBD.

36. The composition of claim 32, which is in the form of a tablet.

37. The composition according to claim 36, wherein the tablet comprises between about 01% and about 2.5% by mass THC.

38. A composition comprising:
at least one cannabinoid comprising D9-THC, D8-THC, THCA, THCV, D8-THCV, D9-THCV, THC VA, CBD, CBDA, CBDV, CBDVA, CBC, CBCA, CBCV, CBCVA, CBG, CBGA, CBGV, CBGVA, CBN, CBNA, CBNV, CBNVA, CBND, CBND A, CBNDV, CBNDVA, CBE, CBEA, CBEV, CBEV A, CBL, CBLA, CBLV, or CBLVA;
a solid powder comprising Microcrystalline Cellulose, Colloidal Silicon Dioxide, Sodium Starch Glycolate, or Sodium Stearyl Fumarate; and
a formulating agent comprising maltodextrin, sorbitol, stevia, mannitol, aspartame, sucralose, isornait, or xylitol.

39. The composition according to claim 38, wherein the solid powder comprises Colloidal Silicon Dioxide.

40. The composition according to claim 39, wherein the formulating agent comprises maltodextrin stevia and xylitol.

41. The composition according to claim 40, which is in the form of a tablet.

42. The composition according to claim 41, wherein the tablet comprises between about 0.1% and about 2.5% by mass THC.

43. A tablet comprising:
at least one cannabinoid comprising THC and CBD;
a solid powder comprising Colloidal Silicon Dioxide;
a formulating agent comprising maltodextrin, stevia and xylitol; and
a flavoring agent,
wherein the tablet comprises between about 0.1% to about 2.5% by mass THC.
